(19) United States
(12) Patent Application Publication
(10) Pub. No.: US 2026/0226013 A1
(43) Pub. Date: Aug. 6, 2026

(54) A PROCESS FOR THE PREPARATION OF PROPAQUIZAFOP

(71) Applicant: CROPNOSYS (INDIA) PRIVATE LIMITED, Bengaluru, Karnataka (IN)

(72) Inventors: Ashok Kumar VELDHANDI, Bengaluru, Karnataka (IN); Rohit GULERIA, Bengaluru, Karnataka (IN)

(73) Assignee: CROPNOSYS (INDIA) PRIVATE LIMITED, Bengaluru, Karnataka (IN)

(21) Appl. No.: 19/152,176

(22) PCT Filed: May 6, 2024
(86) PCT No.: PCT/IB2024/054375

§ 371 Date: Jul. 30, 2025

(30) Foreign Application Priority Data
May 24, 2023 (IN) 202341036012

(51) Int. Cl. C07D 241/44

(52) U.S. Cl. CPC C07D 241/44

(57) ABSTRACT

The present disclosure relates to a process for the preparation of Propaquizafop. The process of the present disclosure is carried out under mild reaction conditions. Further, the process of the present disclosure is a single step synthesis. Furthermore, the process of the present disclosure is simple, cost effective and environment friendly and provides propaquizafop having a comparatively better purity and better yield.

### FIELD

The present disclosure relates to a process for the preparation of propaquizafop.

### BACKGROUND

The background information herein below relates to the present disclosure but is not necessarily prior art.

Propaquizafop is an aryloxyphenoxypropionate herbicide primarily used for post emergence control of weeds. The mode of action of propaquizafop involves inhibition of the plastid acetyl coenzyme A carboxylase (ACCase), which is a key enzyme in fatty acid biosynthesis. Propaquizafop is used for selective weed control in many broadleaf crops such as sugarbeet, rapeseed, soybeans, sunflower, vegetables, fruit trees, vineyards, forestry, and the like. Propaquizafop is quickly absorbed by the leaves and translocated from the foliage to the growing points of the leaves and roots of the sprayed weeds. It is environment friendly and safe to beneficial insects and mammals. Propaquizafop is chemically known as (R)-2-((propan-2-ylideneamino)oxy)ethyl 2-(4-((6-chloroquinoxalin-2-yl)oxy) phenoxy) propanoate. The structure of Propaquizafop is:

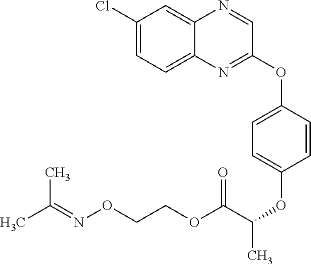 [SMILES: CC(C)=NOCCOC(=O)[C@@H](C)Oc1ccc(Oc2cnc3cc(Cl)ccc3n2)cc1]

Conventional methods for the preparation of Propaquizafop are associated with drawbacks such as having impurities and a low yield of the product. The conventional processes require use of expensive reagents and require further purification which is not economical. The impurities in the final product may affect the efficacy, safety, and stability of the final product.

Therefore, there is felt a need to provide a process for the preparation of Propaquizafop that mitigates the aforestated drawbacks or at least provides a useful alternative.

### OBJECTS

Some of the objects of the present disclosure, which at least one embodiment herein satisfies, are as follows:

It is an object of the present disclosure to ameliorate one or more problems of the background or to at least provide a useful alternative.

Another object of the present disclosure is to provide a process for the preparation of Propaquizafop.

Still another object of the present disclosure is to provide a single step process for the preparation of Propaquizafop.

Yet another object of the present disclosure is to provide a process for the preparation of Propaquizafop with a comparatively better purity and yield.

Still another object of the present disclosure is to provide a simple and cost-effective process for the preparation of Propaquizafop.

Yet another object of the present disclosure is to provide an environment-friendly and commercially scalable process for the preparation of Propaquizafop.

Other objects and advantages of the present disclosure will be more apparent from the following description, which is not intended to limit the scope of the present disclosure.

### SUMMARY

The present disclosure relates to a process for the preparation of Propaquizafop. The process comprising mixing a predetermined amount of alkyl(2R)-2-[4-(6-chloroquinoxalin-2-yl)oxyphenoxy]propanoate in a fluid medium under stirring at a first predetermined temperature to obtain a first mixture. A predetermined amount of a catalyst is added to the first mixture under stirring at the first predetermined temperature to obtain a second mixture. A predetermined amount of 2-[(isopropylideneamino)-oxy]-ethanol is added to the second mixture under stirring at the first predetermined temperature and continuing stirring for a first predetermined time period to obtain a reaction mass. The reaction mass is heated to a second predetermined temperature for a second predetermined time period under stirring to obtain a product mass comprising Propaquizafop.

The alkyl(2R)-2-[4-(6-chloroquinoxalin-2-yl)oxyphenoxy]propanoate is selected from methyl(2R)-2-[4-(6-chloroquinoxalin-2-yl)oxyphenoxy]propanoate and ethyl(2R)-2-[4-(6-chloroquinoxalin-2-yl)oxyphenoxy]propanoate.

The fluid medium is selected from the group consisting of toluene, mesitylene, mix xylene, ortho xylene, para xylene and meta xylene.

The catalyst is selected from the group consisting of titanium (IV) isopropoxide, aluminium trichloride and titanium chloride.

In an embodiment of the present disclosure, a molar ratio of the alkyl(2R)-2-[4-(6-chloroquinoxalin-2-yl)oxyphenoxy]propanoate to the catalyst is in the range of 1:0.05 to 1:0.5.

In an embodiment of the present disclosure, a molar ratio of the alkyl(2R)-2-[4-(6-chloroquinoxalin-2-yl)oxyphenoxy]propanoate to 2-[(isopropylideneamino)-oxy]-ethanol is in the range of 1:1 to 1:5.

The first predetermined temperature is in the range of 20° C. to 50° C.

The first predetermined time period is in the range of 5 minutes to 30 minutes.

The second predetermined temperature is in the range of 120° C. to 160° C.

The second predetermined time period is in the range of 10 hours to 30 hours.

In an embodiment of the present disclosure, a yield of propaquizafop is in the range of 82% to 90% and a purity is in the range of 98% to 99.5%.

In an embodiment of the present disclosure, the product mass is cooled at a temperature in the range of 25° C. to 35° C. followed by adding a predetermined amount of a mineral acid in a dropwise manner under stirring at a third predetermined temperature over a third predetermined time period to obtain a resultant mass and the resultant mass is filtered to obtain a filtrate comprising Propaquizafop.

The third predetermined time period is in the range of 20 minutes to 40 minutes.

The third predetermined temperature is in the range of 5° C. to 20° C.

The mineral acid is selected from the group consisting of hydrochloric acid and sulfuric acid.

### DETAILED DESCRIPTION

The present disclosure relates to a process for the preparation of Propaquizafop.

Embodiments, of the present disclosure, will now be described herein. Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Conventional methods for the preparation of Propaquizafop are associated with drawbacks such as having impurities and a low yield of the product. The conventional processes require further purification which is not economical. The impurities in the final product may affect the efficacy, safety, and stability of the final product.

The present disclosure provides an improved process for the preparation of Propaquizafop.

The process of the present disclosure is simple, environment friendly, economical, and results in improved yield and higher purity of Propaquizafop.

In accordance with the present disclosure, the process for the preparation of Propaquizafop, comprising the following steps:
a) mixing a predetermined amount of alkyl(2R)-2-[4-(6-chloroquinoxalin-2-yl)oxyphenoxy]propanoate in a fluid medium under stirring at a first predetermined temperature to obtain a first mixture;
b) adding a predetermined amount of a catalyst to the first mixture under stirring at the first predetermined temperature to obtain a second mixture;
c) adding a predetermined amount of 2-[(isopropylideneamino)-oxy]-ethanol to the second mixture under stirring at the first predetermined temperature and continuing stirring for a first predetermined time period to obtain a reaction mass; and
d) heating the reaction mass to a second predetermined temperature for a second predetermined time period under stirring to obtain a product mass comprising Propaquizafop.

The process for the preparation of Propaquizafop is described in detail herein below.

In a first step, a predetermined amount of alkyl(2R)-2-[4-(6-chloroquinoxalin-2-yl)oxyphenoxy]propanoate is mixed in a fluid medium under stirring at a first predetermined temperature to obtain a first mixture.

In an embodiment of the present disclosure, alkyl(2R)-2-[4-(6-chloroquinoxalin-2-yl)oxyphenoxy]propanoate is selected from methyl(2R)-2-[4-(6-chloroquinoxalin-2-yl)oxyphenoxy]propanoate and ethyl(2R)-2-[4-(6-chloroquinoxalin-2-yl)oxyphenoxy]propanoate. In an exemplary embodiment of the present disclosure, the alkyl (2R)-2-[4-(6-chloroquinoxalin-2-yl)oxyphenoxy]propanoate is ethyl(2R)-2-[4-(6-chloroquinoxalin-2-yl)oxyphenoxy]propanoate (quizalofop-p-ethyl).

The fluid medium is selected from the group consisting of toluene, mesitylene, mixed xylene, ortho xylene, para xylene and meta xylene. In an exemplary embodiment of the present disclosure, the fluid media is mixed xylene.

The first predetermined temperature is in the range of 20° C. to 50° C. In an exemplary embodiment of the present disclosure, the first predetermined temperature is 30° C.

In a second step, a predetermined amount of a catalyst is added to the first mixture under stirring at the first predetermined temperature to obtain a second mixture.

The catalyst is selected from the group consisting of titanium (IV) isopropoxide, aluminium trichloride and titanium chloride. In an exemplary embodiment of the present disclosure, the catalyst is titanium (IV) isopropoxide

In an embodiment of the present disclosure, a molar ratio of the alkyl(2R)-2-[4-(6-chloroquinoxalin-2-yl)oxyphenoxy]propanoate to the catalyst is in the range of 1:0.05 to 1:0.5. In an exemplary embodiment of the present disclosure, the molar ratio of the alkyl (2R)-2-[4-(6-chloroquinoxalin-2-yl)oxyphenoxy]propanoate to the catalyst is 1:0.1.

In a third step, a predetermined amount of 2-[(isopropylideneamino)-oxy]-ethanol is added to the second mixture under stirring at the first predetermined temperature and continuing stirring for a first predetermined time period to obtain a reaction mass.

The first predetermined time period is in the range of 5 minutes to 30 minutes. In an exemplary embodiment of the present disclosure, the first predetermined time period is 10 minutes.

In an embodiment of the present disclosure, a molar ratio of the alkyl(2R)-2-[4-(6-chloroquinoxalin-2-yl)oxyphenoxy]propanoate to 2-[(isopropylideneamino)-oxy]-ethanol is in the range of 1:1 to 1:5. In an exemplary embodiment of the present disclosure, the molar ratio of the alkyl(2R)-2-[4-(6-chloroquinoxalin-2-yl)oxyphenoxy]propanoate to 2-[(isopropylideneamino)-oxy]-ethanol is 1:1.3.

In a fourth step, the reaction mass is heated to a second predetermined temperature for a second predetermined time period under stirring to obtain a product mass comprising Propaquizafop.

The second predetermined temperature is in the range of 120° C. to 160° C. In an exemplary embodiment of the present disclosure, the second predetermined temperature is 140° C.

The second predetermined time period is in the range of 10 hours to 30 hours. In an exemplary embodiment of the present disclosure, the second predetermined time period is 16 hours.

In an embodiment of the present disclosure, as the reaction proceeds there are formation of by-products such as ethanol, methanol, titanium hydroxide and/or titanium dioxide, S-isomer of propaquizafop, quizalofop-(p)-isopropyl ester, and quizalofop-(p)-methyl ester.

In an embodiment of the present disclosure, the product mass is cooled at a temperature in the range of 25° C. to 35° C. followed by adding a predetermined amount of a mineral acid in a dropwise manner under stirring at a third predetermined temperature over a third predetermined time period to obtain a resultant mass. The resultant mass is filtered to obtain a filtrate comprising Propaquizafop.

The third predetermined time period is in the range of 20 minutes to 40 minutes. In an exemplary embodiment of the present disclosure, the third predetermined time period is 30 minutes.

The third predetermined temperature is in the range of 5° C. to 20° C. In an exemplary embodiment of the present disclosure, the third predetermined temperature is 10° C.

The mineral acid is selected from the group consisting of hydrochloric acid and sulfuric acid. In an exemplary embodiment of the present disclosure, the mineral acid is hydrochloric acid.

In an embodiment of the present disclosure, the filtrate is allowed to settle for a time period in the range of 20 minutes to 40 minutes so as to separate out two layers to obtain an organic layer and an aqueous layer.

In an embodiment of the present disclosure, the organic layer is separated and washed with 10% NaHCO₃ solution followed by washing with water to obtain a washed organic layer. The washed organic layer is distilled under reduced pressure at a temperature in the range of 80° C. to 95° C. to obtain a crude solid product, propaquizafop. Isopropyl alcohol is added to the crude propaquizafop and stirred for a time period in the range of 20 minutes to 40 minutes followed by filtration to obtain pure propaquizafop.

In accordance with the process of the present disclosure, propaquizafop so obtained has a yield in the range of 82% to 90% and a purity in the range of 98% to 99.5%. In an exemplary embodiment of the present disclosure the yield of propaquizafop is 88% and the purity is 99%.

In accordance with an exemplary embodiment of the present disclosure, the schematic representation for the preparation of Propaquizafop is illustrated as below scheme I:

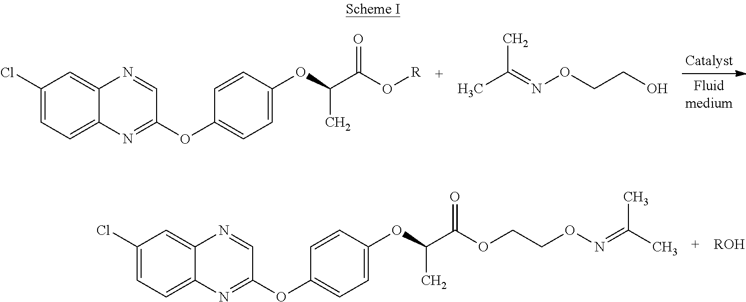

wherein R is selected from methyl and ethyl.

The present disclosure provides a simple, environment friendly and economic process for the preparation of propaquizafop with a comparatively better yield and better purity.

The foregoing description of the embodiments has been provided for purposes of illustration and is not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to the industrial scale.

### EXPERIMENTAL DETAILS

### Example 1: Preparation of Propaquizafop from ethyl(2R)-2-[4-(6-chloroquinoxalin-2-yl)oxyphenoxy]propanoate in Accordance with the Present Disclosure

A reactor was charged with 700 g of mixed Xylene (3 vol) followed by adding 100 g of ethyl (2R)-2-[4-(6-chloroquinoxalin-2-yl)oxyphenoxy]propanoate at 30° C. under stirring to obtain a first mixture. 7.6 g of titanium (IV) isopropoxide was added to the first mixture at 30° C. under stirring to obtain a second mixture. 40.85 g of 2-[(isopropylideneamino)-oxy]-ethanol (IPAE) was added to the second mixture at 30° C. under stirring and was further stirred at 30° C. for 10 minutes to obtain a reaction mass. The reaction mass was heated to 140° C. under stirring and maintaining for 16 hours to obtain a product mass comprising Propaquizafop. During the reaction, ethanol (by-product) was collected in the receiver. The progress of reaction was monitored by HPLC/TLC.

The product mass comprising propaquizafop was cooled to 25° C. under stirring to obtain a cooled mass. 35% HCl was added dropwise to the cooled mass over a period of 30 minutes at 10° C. to obtain a resultant mass. The resultant mass was filtered through a Celite bed to obtain a filtrate comprising Propaquizafop. The filtrate was allowed to settle for 30 minutes as to separate out the two layers to obtain an organic layer and an aqueous layer. The organic layer was separated and washed with 10% NaHCO₃ solution followed by water to obtain a washed organic layer. The washed organic layer was distilled under reduced pressure at 90° C. to obtain a crude solid product, propaquizafop.

40 ml of isopropyl alcohol was added to the crude propaquizafop and stirred for 30 minutes at 30° C. followed by filtration to obtain pure propaquizafop which was washed with isopropyl alcohol and dried at 50° C. for 5 hours to obtain crystals of propaquizafop (Yield: 88% and Purity: 99%).

### COMPARATIVE EXAMPLES

### Comparative Example 1 (Conventional Process for the Preparation of Propaquizafop)

In a first step, (S)-2-(4-((6-chloroquinoxalin-2-yl)oxy) phenoxy) propanoic acid was chlorinated by using thionyl chloride in methylene dichloride (MDC) at 40° C. to obtain(S)-2-(4-((6-chloroquinoxalin-2-yl)oxy) phenoxy) propanoyl chloride.

In a second step, (S)-2-(4-((6-chloroquinoxalin-2-yl)oxy) phenoxy) propanoyl chloride was reacted with propan-2-one O-(2-hydroxyethyl) oxime by using potassium carbonate as a base in mix xylene at 140° C. to obtain propaquizafop having yield 80% and purity 95%.

The schematic representation for the preparation of Propaquizafop in accordance with the comparative example 1 is illustrated below as scheme II:

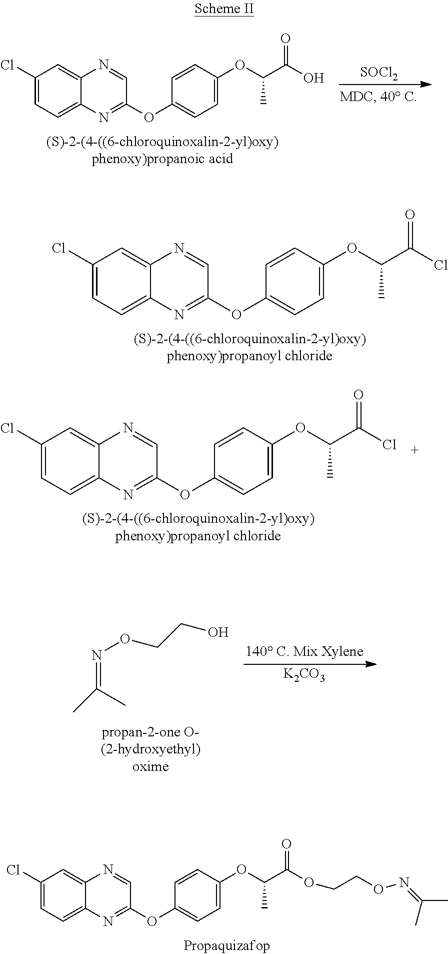

### Comparative Example 2 (Conventional Process for the Preparation of Propaquizafop)

In a first step, 4-methylbenzene-1-sulfonylchloride was reacted with propan-2-one-O-(2-hydroxyethyl) oxime in the presence of tetrabutylammonium bromide (TBAB) to obtain 2-((propan-2-ylideneamino)oxy)ethyl 4-methylbenzenesulfonate.

In a second step, 2-((propan-2-ylideneamino)oxy)ethyl 4-methylbenzenesulfonate was reacted with(S)-2-(4-((6-chloroquinoxalin-2-yl)oxy) phenoxy) propanoic acid by using potassium carbonate as a base in mix xylene at 140° C. to obtain propaquizafop having yield 75% and purity 95%.

The schematic representation for the preparation of Propaquizafop in accordance with the comparative example 2 is illustrated below as scheme III:

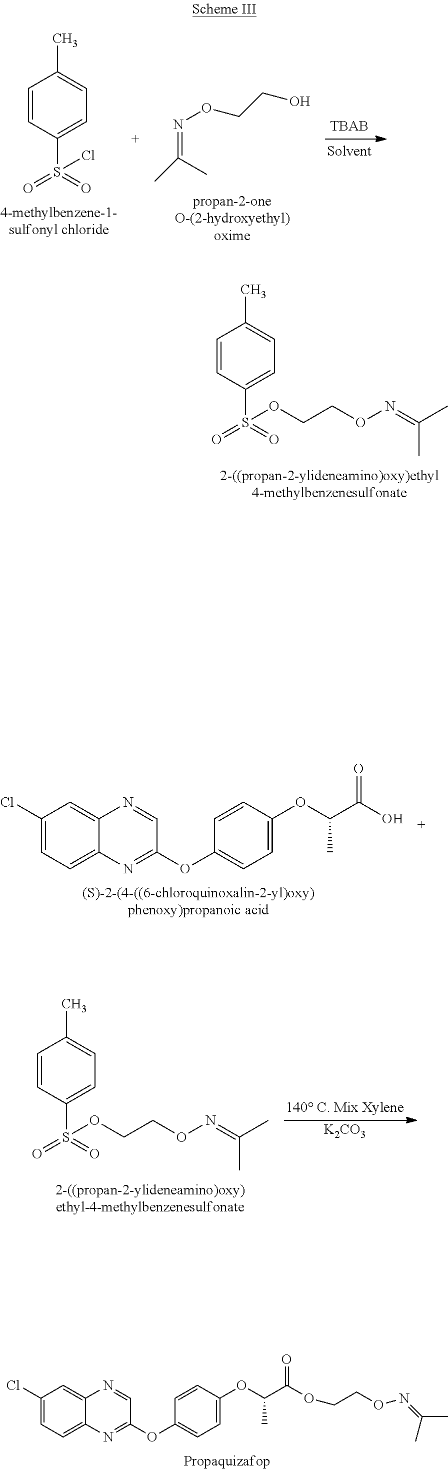

From the example 1 and the comparative examples 1 and 2, it is observed that the process for the preparation of Propaquizafop in accordance with the present disclosure is a single pot process with minimum reagents in minimum amount (catalytic amount only) and the final product Propaquizafop obtained is with comparatively better yield and better purity with less formation of by-products. Further, the reaction operations in the process of the present disclosure are easy and involve simple work up procedure and isolation of the product. Furthermore, the fluid media used in the process of the present disclosure can be recovered and re-used in subsequent batches.

In ethyl(2R)-2-[4-(6-chloroquinoxalin-2-yl)oxyphenoxy]propanoate, (2R) represents R enantiomer; and

In(S)-2-(4-((6-chloroquinoxalin-2-yl)oxy) phenoxy) propanoic acid, (S) represents S enantiomer.

R/S system is the nomenclature system for enantiomers. In the R/S nomenclature system, each chiral center is assigned R or S configuration based on the substituents of the chiral center (R and S refer to rectus and sinister, Latin for right and left, respectively). According to this nomenclature, substituents on the chiral center are each assigned a priority based on atomic number, following the Cahn-Ingold-Prelog rules. After assigning the priority to the substituents, if the arrow rotates clockwise then it is R configuration. After assigning the priority to the substituents, if the arrow rotates anti-clockwise then it is S configuration.

### TECHNICAL ADVANCEMENT

The present disclosure described hereinabove has several technical advantages including, but not limited to, the realization of a process for the preparation of propaquizafop that;
is carried out under mild reaction conditions;
is a single step synthesis;
is simple, cost effective and environment friendly; and
provides propaquizafop having a comparatively better purity and yield.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values given for various physical parameters, dimensions, and quantities are only approximate values and it is envisaged that the values higher than the numerical value assigned to the physical parameters, dimensions, and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A process for the preparation of Propaquizafop, said process comprising the following steps:
a) mixing a predetermined amount of alkyl(2R)-2-[4-(6-chloroquinoxalin-2-yl)oxyphenoxy]propanoate in a fluid medium under stirring at a first predetermined temperature to obtain a first mixture;
b) adding a predetermined amount of a catalyst to said first mixture under stirring at said first predetermined temperature to obtain a second mixture;
c) adding a predetermined amount of 2-[(isopropylideneamino)-oxy]-ethanol to said second mixture under stirring at said first predetermined temperature and continuing stirring for a first predetermined time period to obtain a reaction mass; and
d) heating said reaction mass to a second predetermined temperature for a second predetermined time period under stirring to obtain a product mass comprising Propaquizafop.

2. The process as claimed in claim 1, wherein said alkyl(2R)-2-[4-(6-chloroquinoxalin-2-yl)oxyphenoxy]propanoate is selected from methyl(2R)-2-[4-(6-chloroquinoxalin-2-yl)oxyphenoxy]propanoate and ethyl(2R)-2-[4-(6-chloroquinoxalin-2-yl)oxyphenoxy]propanoate.

3. The process as claimed in claim 1, wherein said fluid medium is selected from the group consisting of toluene, mesitylene, mix xylene, ortho xylene, para xylene and meta xylene.

4. The process as claimed in claim 1, wherein said catalyst is selected from the group consisting of titanium (IV) isopropoxide, aluminium trichloride and titanium chloride.

5. The process as claimed in claim 1, wherein a molar ratio of said alkyl(2R)-2-[4-(6-chloroquinoxalin-2-yl)oxyphenoxy]propanoate to said catalyst is in the range of 1:0.05 to 1:0.5.

6. The process as claimed in claim 1, wherein a molar ratio of said alkyl(2R)-2-[4-(6-chloroquinoxalin-2-yl)oxyphenoxy]propanoate to 2-[(isopropylideneamino)-oxy]-ethanol is in the range of 1:1 to 1:5.

7. The process as claimed in claim 1, wherein said first predetermined temperature is in the range of 20° C. to 50° C.

8. The process as claimed in claim 1, wherein said first predetermined time period is in the range of 5 minutes to 30 minutes.

9. The process as claimed in claim 1, wherein said second predetermined temperature is in the range of 120° C. to 160° C.

10. The process as claimed in claim 1, wherein said second predetermined time period is in the range of 10 hours to 30 hours.

11. The process as claimed in claim 1, wherein a yield of propaquizafop is in the range of 82% to 90% and a purity is in the range of 98% to 99.5%.

12. The process as claimed in claim 1, wherein said product mass is cooled at a temperature in the range of 25° C. to 35° C. followed by adding a predetermined amount of a mineral acid in a dropwise manner under stirring at a third predetermined temperature over a third predetermined time period to obtain a resultant mass and the resultant mass is filtered to obtain a filtrate comprising Propaquizafop.

13. The process as claimed in claim 12, wherein said third predetermined time period is in the range of 20 minutes to 40 minutes.

14. The process as claimed in claim 12, wherein said third predetermined temperature is in the range of 5° C. to 20° C.

15. The process as claimed in claim 12, wherein said mineral acid is selected from the group consisting of hydrochloric acid and sulfuric acid.
